(19) United States
(12) Patent Application Publication
(10) Pub. No.: US 2025/0257303 A1
(43) Pub. Date: Aug. 14, 2025

(54) A CLOSURE FOR A BIOREACTOR

(71) Applicant: Global Life Sciences Solutions USA LLC, Marlborough, MA (US)

(72) Inventor: Eric FALDT, Uppsala (SE)

(73) Assignee: Global Life Sciences Solutions USA LLC, Marlborough, MA (US)

(21) Appl. No.: 18/992,251

(22) PCT Filed: Jun. 26, 2023
(86) PCT No.: PCT/US2023/026182

§ 371 Date: Jan. 8, 2025

(30) Foreign Application Priority Data
Jul. 14, 2022 (GB) 2210354.3

(51) Int. Cl. C12M 1/04; C12M 1/00; C12M 1/34

(52) U.S. Cl. CPC C12M 27/04; C12M 29/08; C12M 41/26; C12M 41/30

(57) ABSTRACT

A closure 1; 1′ for a bioreactor 200; 200′; 200″, said closure being configured for being connectable to a container 100; 100′; 100″ such that the closure 1; 1′ and the container 100; 100′; 100″ together constitutes the bioreactor 200; 200′; 200″, wherein the closure 1; 1′ comprises an impeller device 11 comprising an impeller shaft 13 and impeller blades 15 mounted to the impeller shaft 13, wherein said impeller device 11 is mounted to the closure 1; 1′ such that the impeller shaft 13 and the impeller blades 15 protrude into the container 100; 100′; 100″ when the closure 1; 1′ is connected to the container 100; 100′; 100″ and a gas delivery device 21 which is arranged in the closure 1; 1′ such that it passes from a gas inlet 23 at an outside surface 3b of the closure 1; 1′ to an inside surface 3a of the closure 1; 1′ and protrudes from the inside surface 3a such that a gas outlet 25 of the gas delivery device is provided inside the container 100; 100′; 100″ when the closure 1; 1′ is connected to the container 100; 100′; 100″.

### CROSS-REFERENCE TO RELATED APPLICATIONS

This application is a national stage of International Application No. PCT/US2023/026182 filed on Jun. 26, 2023, which claims the benefit of Great Britian Application No. 2210354.3, filed on Jul. 14, 2022, all of which are hereby incorporated by reference herein in their entireties.

### TECHNICAL FIELD OF THE INVENTION

The present invention relates to a closure for a bioreactor, a bioreactor, and to a method for transforming a container into a bioreactor.

### BACKGROUND

For process development applications in different fields of research small-scale bioreactors are sometimes needed. Often, different sizes of the bioreactors are needed for different stages of a development process.

Shake flasks may be used for small scale cultivation, such as for volumes between 25-250 mL. However, this is an uncontrolled process. Ambr® 15 and Ambr® 250 from Sartorius are two examples of controllable small-scale bioreactors. However, these are expensive and for some applications other sizes of bioreactors may be needed.

### SUMMARY

An object of the present invention is to provide a flexible small-scale bioreactor.

A further object of the present invention is to provide a method for transforming a container into a bioreactor.

This is achieved by a closure for a bioreactor, by a bioreactor, and by a method according to the independent claims.

According to one aspect of the invention, a closure for a bioreactor is provided, said closure configured to be connected to a container such that the closure and the container together constitutes the bioreactor. The closure comprises an inside surface which will face an inside of the bioreactor when the closure is connected to the container and an outside surface which will form a part of an outer surface of the bioreactor when the closure is connected to the container.

The closure may comprise: (i) an impeller device comprising an impeller shaft and impeller blades mounted to the impeller shaft, wherein said impeller device is mounted to the inside surface of the closure such that the impeller shaft and the impeller blades protrude into the container when the closure is connected to the container; and (ii) a gas delivery device comprising a gas inlet, a gas outlet and a gas delivery channel reaching between said gas inlet and said gas outlet, wherein said gas inlet is provided at the outside surface of the closure and said gas delivery channel is arranged in the closure such that it passes from the gas inlet at the outside surface of the closure to the inside surface of the closure and protrudes from the inside surface such that the gas outlet of the gas delivery channel is provided inside the container when the closure is connected to the container.

According to another aspect of the invention, a bioreactor comprising a container connected to a closure according to the invention is provided.

According to another aspect of the invention, a method for transforming a container into a bioreactor is provided, said method comprising the step of attaching a closure according to the invention to the container hereby providing a bioreactor.

Hereby a closure, such as a cap or a lid, is provided which can be used together with a container for providing a bioreactor. The container does not need to be adapted in any specific way for being a bioreactor. The container only needs to be compatible with the closure such that they can be connected to each other. Furthermore, the container needs to be made from a suitable material which can be sterilized and provide a sterile environment for a bioreactor culture to be kept in the bioreactor. An impeller device and a gas delivery device are each provided in the closure such that the container does not need to include or have integrated there such parts. Suitably, all the features which are necessary for cell culture are included in the closure, and thus there is no need for any specific adaptations or any bioreactor features in the container itself. Any container can be used with the closure, as a bioreactor, as long as it can be connected to the closure. Different sizes of containers can be used together with the same closure as long as the containers can be connected to the closure. By providing such a design and arrangement, a flexible and cost-efficient bioreactor is achieved. For example, the user can utilize containers which they already have. The container can be a bottle of any size, for example having an internal volume between 250 mL-2 L. Furthermore, the closure and bioreactor according to the invention is suitable for providing a closed and sterile handling environment for culturing cells.

In one embodiment of the invention, the impeller shaft is telescopic such that a length of the impeller shaft can be adjusted. The adjustability allows the closure to be used with containers of different sizes (e.g., heights).

In one embodiment of the invention, the impeller shaft comprises a hollow outer shaft, which is connected to the inside surface of the closure, and an inner shaft that is telescopically arranged inside the hollow outer shaft such that it can protrude from the hollow outer shaft to different amounts. The inner shaft is connected to the impeller blades and the inner shaft is mounted to the hollow outer shaft such that it can rotate around a longitudinal central axis, A, of the impeller shaft, thereby mixing the contents of the bioreactor.

In one embodiment of the invention, the gas delivery channel of the gas delivery device is provided inside the impeller device. A gas outlet of the gas delivery device can be provided at the end of the impeller device such that gas can be delivered into the bioreactor close to a bottom of the bioreactor, which promotes gas distribution.

In one embodiment of the invention, the closure further comprises a sensor arrangement comprising at least one sensor. The sensor arrangement is arranged in the closure such that the at least one sensor is provided inside the container when the closure is connected to the container.

In one embodiment of the invention, the sensor arrangement comprises at least one sensor accessing channel, the at least one sensor accessing channel arranged in the closure such that it passes from the outside surface of the closure to the inside surface of the closure and protrudes from the inside surface of the closure. The at least one sensor is arranged in the at least one sensor accessing channel such that the at least one sensor can be used for measuring a feature of a content provided in the bioreactor when the closure is connected to the container and such that the sensor is accessible from the outside surface of the closure via the sensor accessing channel.

In one embodiment of the invention, the at least one sensor comprises a pH, dissolved oxygen (DO), and/or temperature sensor. The at least one sensor may also comprise a imaging or optical sensor for determining, for example, cell density, viable cell density, analyte concentrations, etc. The sensor may be an optic sensor or a chemo electrical sensor.

In one embodiment of the invention, the gas inlet is configured for connection to an external gas source.

In one embodiment of the invention, the impeller device comprises at least one magnetic element, such that the impeller can be driven (i.e., rotated) by application of an external magnetic field from outside the container when the closure is connected to the container.

In one embodiment of the invention, the closure further comprises a securing mechanism for securing the closure to the container.

In one embodiment of the invention, the closure is configured for being connected to a container that is a bottle.

In one embodiment of the invention the closure is configured for being connected to a container having an inner volume of between 50 mL-2 L.

### BRIEF DESCRIPTION OF THE DRAWINGS

FIGS. 1a-1c are different schematic views of a closure, according to one embodiment of the invention.

FIG. 2a shows schematically a closure, according to one embodiment of the invention, connected to a container together making up a bioreactor.

FIG. 2b shows schematically a closure, according to one embodiment of the invention, connected to a container together making up a bioreactor.

FIG. 2c shows schematically a closure, according to one embodiment of the invention, connected to a container together making up a bioreactor.

FIG. 3 is a flow chart of a method according to one embodiment of the invention.

### DETAILED DESCRIPTION OF EMBODIMENTS

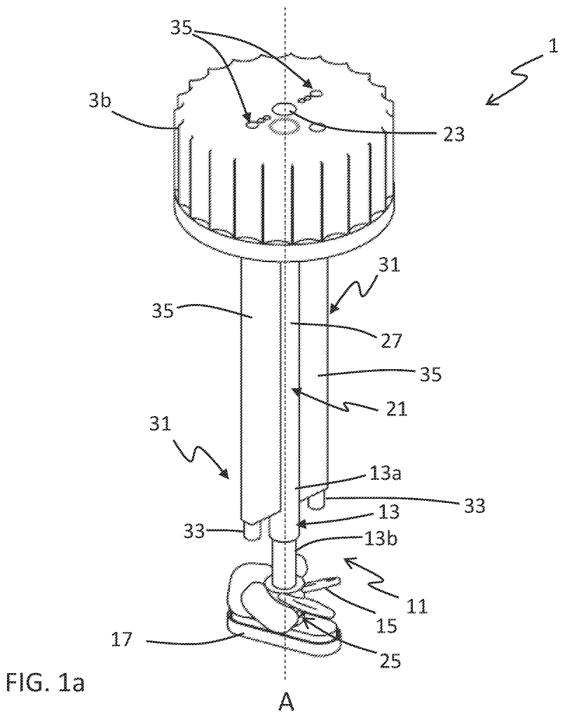

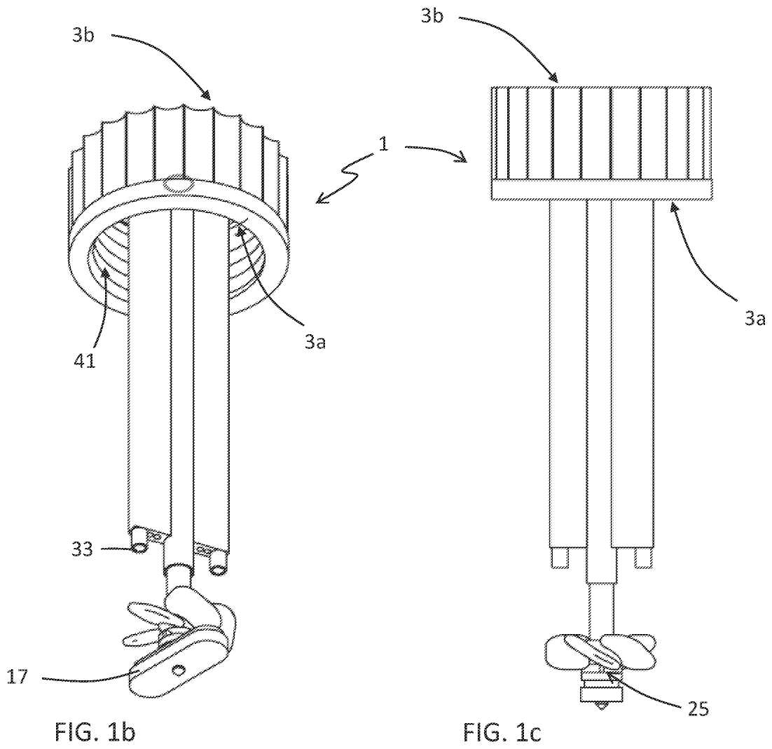

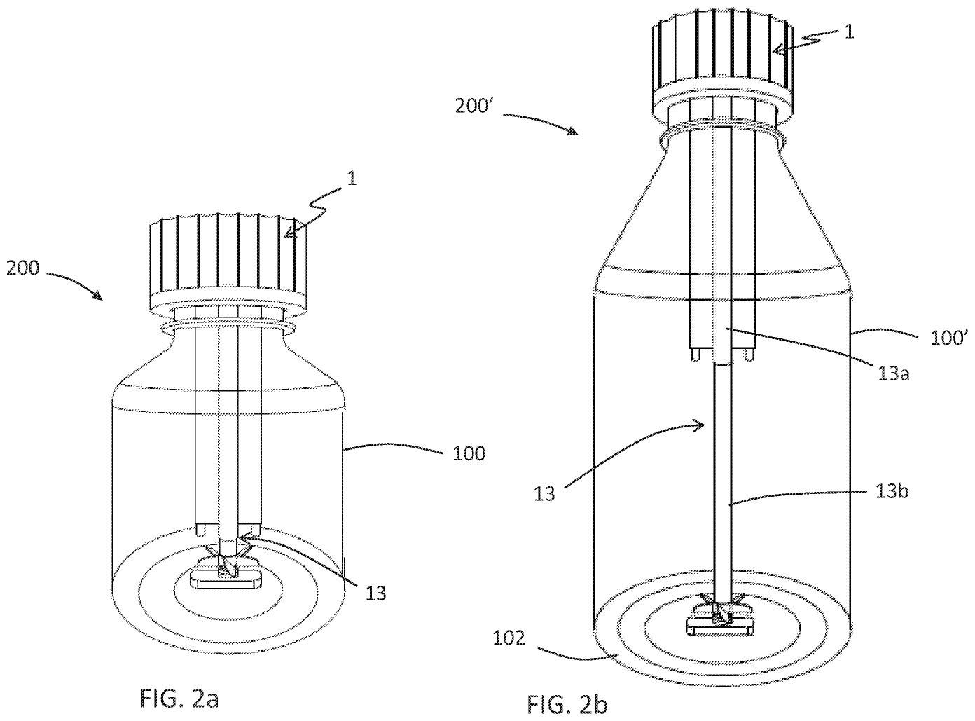

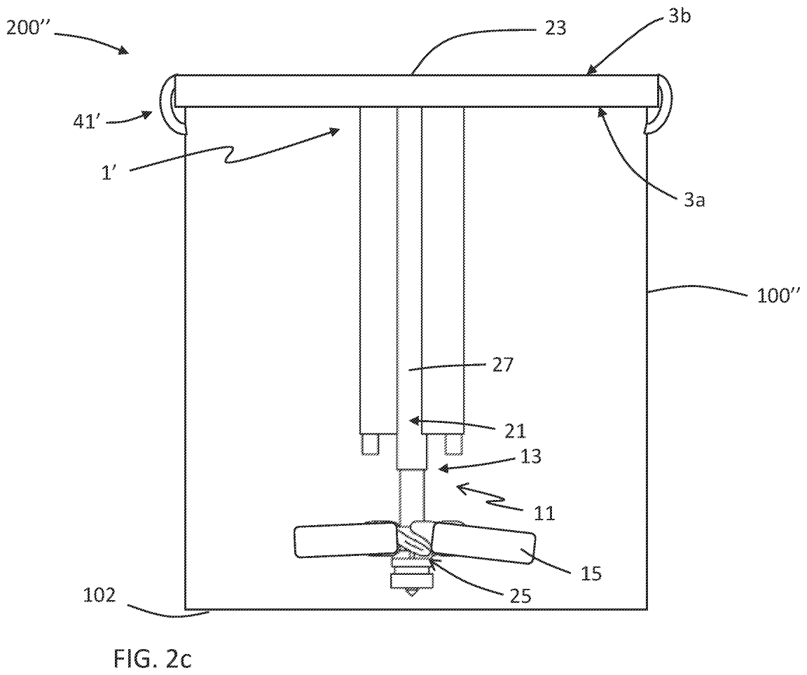

According to the invention a closure 1; 1′ for a bioreactor and a bioreactor 200; 200′; 200″ comprising such a closure 1; 1′ are provided. In FIGS. 1a-1c one embodiment of a closure 1 according to the invention is shown. In FIGS. 2a-2c different embodiments of bioreactors 200; 200′; 200″ according to the invention are shown. In FIGS. 2a and 2b bioreactors 200; 200′ comprising the closure 1 as shown in FIGS. 1a-1c are shown. In FIG. 2c a bioreactor 200″ comprising a different embodiment of a closure 1′ according to the invention is shown. Many of the details of the embodiments of the closure 1; 1′ are the same or similar and are given the same or similar reference numbers and the description thereof may only be provided for one, but is explicitly applicable to all embodiments unless specified otherwise in this text.

The closure 1; 1′ according to the invention is configured for being connectable to a container 100; 100′; 100″ such that the closure 1; 1′ and the container 100; 100′; 100″ together constitutes a bioreactor 200; 200′; 200″. The closure 1; 1′ comprises an inside surface 3a which will face an inside of the bioreactor 200; 200′; 200″ when the closure 1; 1′ is connected to the container 100; 100′; 100″ and an outside surface 3b which will form a part of an outer surface of the bioreactor 200; 200′; 200″ when the closure 1; 1′ is connected to the container 100; 100′; 100″. The closure 1; 1′ can be, for example, a cap or a lid and can be connected to the container 100; 100′; 100″ by any suitable securing mechanism 41; 41′ (e.g., threading, a clamp, latch, etc.). In the embodiment of the closure 1 as shown in FIGS. 1a-1c, the securing mechanism 41 is cooperating threads on container 100; 100′ and closure 1. Another example of a securing mechanism 41′ comprising a latching mechanism is shown in FIG. 2c. A magnetic locking is another example of a securing mechanism which can be used. The container 100; 100′; 100″ can be, for example, a bottle, a jar, a box or another type of container. The same closure 1; 1′ can, in some examples, be connected to containers 100; 100′; 100″ of different sizes. This is shown in FIGS. 2a and 2b where the same closure 1 is connected to two different sizes of containers 100; 100′ in the form of bottles having different heights and volumes. This invention is especially suitable for process development and therefore an inner volume of the containers 100; 100′; 100″ can suitably range between, for example, 50 mL-2 L. A closed and sterile handling of the bioreactor culture is possible thanks to the closure and bioreactor according to the invention. The closure can, for example, be presterilized or it can be sterilized together with the container in an autoclaving process.

The closure 1; 1′ comprises an impeller device 11 comprising an impeller shaft 13 and impeller blades 15 mounted to the impeller shaft 13. Said impeller device 11 is mounted to the inside surface 3a of the closure 1; 1′ such that the impeller shaft 13 and the impeller blades 15 protrude into the container 100; 100′; 100″ when the closure 1; 1′ is connected to the container 100; 100′; 100″. The closure 1; 1′ further comprises a gas delivery device 21 comprising a gas inlet 23, a gas outlet 25 and a gas delivery channel 27 reaching between said gas inlet 23 and said gas outlet 25. The gas inlet 23 is provided at the outside surface 3b of the closure 1; 1′ and said gas delivery channel 27 is arranged in the closure 1; 1′ such that it passes from the gas inlet 23 at the outside surface 3b of the closure 1; 1′ to the inside surface 3a of the closure 1 and protrudes from the inside surface 3a such that the gas outlet 25 of the gas delivery channel 22 is provided inside the container 100; 100′; 100″ when the closure 1; 1′ is connected to the container 100; 100′; 100″. The gas inlet 23 can be configured for connection to an external gas source.

According to embodiments, the impeller shaft 13 is telescopic, such that a length of the impeller shaft 13 can be adjusted. As shown in the Figures and discussed in greater detail below, the impeller shaft 13 includes a hollow outer shaft 13a which is connected to the inside surface 3a of the closure 1; 1′ and an inner shaft 13b which is telescopically arranged inside the hollow outer shaft 13a such that it can protrude from the hollow outer shaft 13a to different lengths. This is the case for both the embodiments of closures 1; 1′ as shown in FIGS. 1 and 2. However, in another embodiment the impeller shaft 13 could have a fixed length. The benefit of having a telescopic impeller shaft 13 is that a length of the impeller shaft 13 can be adjusted for different sizes of containers 100; 100′; 100″. This is shown in FIGS. 2a, 2b and 2c where the impeller shaft 13 has been extended different amounts. The impeller blades 15 can be provided close to a bottom 102 of the container 100; 100′; 100″ which often is suitable. The bioreactor can be filled with media to different amounts. Furthermore, the gas outlet 25 of the gas delivery device 21, if integrated with the impeller device 11, as will be further described below, can also be provided close to the bottom 102 of the container 100; 100′; 100″ so that gas can be bubbled through a substantial portion of the media.

In some embodiments, the impeller blades 15 are integrally attached to the impeller. In further embodiments, the impeller blades can be attached to the impeller by way of a hinge, such that the impeller blades 15 can be folded up toward the impeller shaft 13. By attaching the impeller blades to the impeller 11 with hinges the length of the blades 15 can be increased while still allowing them to pass through the opening in the container 100; 100′; 100″. This is particularly beneficial when the container has a small opening but a larger volume capacity where larger impeller blades 15 are necessary in order to ensure proper mixing.

In some embodiments, (as shown in both embodiments of closures 1; 1′ as illustrated in FIGS. 1 and 2) the impeller shaft 13 comprises a hollow outer shaft 13a connected to the inside surface 3a of the closure 1; 1′ and an inner shaft 13b which is telescopically arranged inside the hollow outer shaft 13a such that it can protrude from the hollow outer shaft 13a to different lengths. Telescopically as used here should mean that the inner shaft 13b can slide with relation to the outer shaft 13a. The inner shaft 13b is connected to the impeller blades 15 and said inner shaft 13b is mounted to the hollow outer shaft 13a such that it can rotate around a longitudinal central axis, A, of the impeller shaft 13. The impeller shaft 13 can, in some embodiments, automatically adjust to the size of the container 100; 100′; 100″ (e.g., by gravity pulling the inner shaft 13b down). The inner shaft 13b and the hollow outer shaft 13a are suitably concentrically arranged. The inner shaft 13b can have a cylindrical form and the hollow outer shaft 13a can have a hollow cylindric form. An o-ring or some other type of bearing element may be provided between the inner and outer shafts 13a, 13b so that the outer shaft 13a and inner shaft 13b maintain a consistent concentric orientation (e.g., the inner shaft 3b cannot take an angled orientation relative to the outer shaft 13a). However, this may not be necessary. Another alternative can be to use hydrophobic and/or hydrophilic materials for the inner and outer shafts 13a, 13b whereby leakage can be avoided without any mechanical sealing.

The impeller device 11 comprises, in some embodiments, a magnetic element 17 for allowing rotation of the impeller device 11 around the longitudinal central axis, A, from outside the container 100; 100′; 100″ when the closure 1; 1′ is connected to the container 100; 100′; 100′ by application of an external magnetic field. The magnetic part 17 can be provided at the end of the inner shaft 13b as shown in both embodiments of the closure 1; 1′ as illustrated in FIGS. 1 and 2. Hereby the magnetic part 17 will be provided close to the bottom 102 of the container 100; 100′; 100″ when the closure 1; 1′ is connected to the container 100; 100′; 100″ and as such, a magnetic drive from below the bottom 102 of the container 100; 100′; 100″ can be implemented in order to rotate the impeller to effectuate mixing of fluid in the bioreactor 1; 1′. Other possibilities for driving the impeller can be for example an electric motor connected to the impeller shaft 13 from the outside surface 3b of the closure 1; 1′.

In embodiments, (and as shown for both the embodiments of closures 1; 1′ as illustrated in FIGS. 1 and 2) the gas delivery channel 27 of the gas delivery device 21 is provided inside the impeller device 11. The telescopic impeller shaft 13 can, as described above, be hollow such that the gas delivery channel 27 can be provided through the whole impeller shaft 13. The gas outlet 25 can be provided at the end of the impeller shaft 13, underneath the impeller blades 15, and gas can be delivered into the bioreactor 200; 200′; 200″ from there. Delivery of gas is hereby provided close to the bottom 102 of the container 100; 100′; 100′ of the bioreactor 200; 200′; 200″ and directly below the impeller blades 15 which is suitable for good gas distribution in the content of the bioreactor. This is however not necessary for all embodiments. In another embodiment the gas delivery device 21 can be a separate device.

The closure 1; 1′ can be made at least partly from a plastic material or possibly from a metal. The closure should be suitable for sterilization.

In some embodiments of the invention, (as shown for both embodiments of closures 1; 1′ illustrated in FIGS. 1 and 2) the closure 1; 1′ further comprises a sensor arrangement 31 comprising one or more sensors 33. The closure 1; 1′ may comprise more than one sensor arrangements 31. The sensor arrangement 31 is arranged in the closure 1; 1′ such that the one or more sensors 33 are provided inside the container 100; 100′; 100″ when the closure 1; 1′ is connected to the container 100; 100′; 100″. The sensor arrangement 31 may comprise one or more sensor accessing channels 35, wherein said sensor accessing channels 35 are arranged in the closure 1; 1′ such that they pass from the outside surface 3b of the closure 1; 1′ to the inside surface 3a of the closure 1; 1′ and protrude from the inside surface 3a of the closure 1; 1′. The one or more sensors 33 are arranged in the sensor accessing channels 35 such that the sensors 33 can be used for measuring features of a content provided in the bioreactor when the closure 1; 1′ is connected to the container 100; 100′; 100″ and such that the one or more sensors 33 are accessible from the outside surface 3b of the closure 1; 1′ via the sensor accessing channel 35. Depending on sensor technique and depending on what is measured the sensor may need to be in direct contact with the bioreactor culture or not. For some types of sensors, the sensor needs to be in direct contact with the content in the bioreactor. For such sensors, the sensor is mounted to the sensor accessing channel such that it is in direct contact with the content in the bioreactor and such that it can be connected to from outside the bioreactor via the sensor accessing channel 35. For other types of sensors, such as optic sensors, direct contact with the content is not needed, but measuring can be performed through a transparent wall. In some embodiments of the invention, the sensor accessing channel 35 and the impeller shaft 13 are connected to one another to form a baffle. This may be advantageous for providing better mixing in the bioreactor because fluid flows induced by the impeller will be disrupted whereby better mixing is achieved. However, in other embodiments the one or more sensor accessing channels 35 can be separated from the impeller shaft 13. A baffle can also be arranged in another way in the bioreactor according to the invention. For example, a hinged baffle can be provided in connection with the impeller device which baffle can be automatically folded out by the vortex.

In some embodiments of the invention, the sensor 33 comprises a pH sensor and/or a DO sensor. Other sensors can also be provided such as a temperature sensor and/or an imaging or optical sensor for determining, for example, cell density, viable cell density, analyte concentrations. The sensor 33 can for example be an optic sensor or a chemo electrical sensor. Other types of sensors such as resistive or colorimetric sensors can also be provided. For an optical sensor, an optic fiber is provided through the sensor accessing channel 35 for connection to the optic sensor. Any number of sensors 33 and sensor accessing channels 35 can be provided. More than one sensor 33 can also be provided in connection with the same sensor accessing channel 35.

In some embodiments, the closure 1; 1′ further comprises one or more access channels 36 for sampling and/or addition of ingredients to the bioreactor content, to effectuate, for example, addition of fresh culture media, to carry out a perfusion operation, for washing, and/or for aseptic sampling. Two or more such access channels can possible be used for a loop for continuous measuring of for example carbon source (such as glucose) and/or metabolites (such as lactate). Two access channels could also be used for running the bioreactor in fed batch or perfusion mode.

According to the invention a bioreactor 200; 200′; 200″ is also provided. The bioreactor 200; 200′; 200″ comprises a closure 1; 1′ as described above according to the invention and a container 100; 100′; 100″ connected to each other. All the bioreactor features, such as the impeller device 11, the gas delivery device 21 and the sensor arrangement 31 are connected to the closure 1; 1′ whereby the container can be an empty container which does not comprise any bioreactor features, such as a normal bottle. The container may be made of a material that is light-blocking so that culture media which is sensitive to light can be utilized.

In embodiments, the bioreactor 200; 200′; 200″ may further include at least one baffle. The baffle can take the form of a flexible insert that can be place inside the container 100; 100′; 100″ and line the inner wall thereof. In one example, the baffle comprises a flexible ring-shaped insert that includes at least one protrusion.

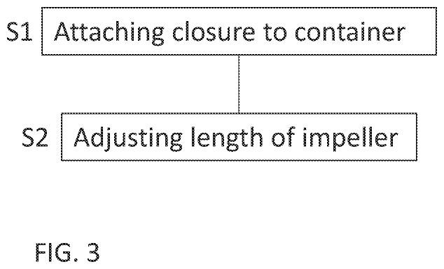

According to the invention, a method for transforming a container 100; 100′; 100″ into a bioreactor 200; 200′; 200″ is also provided. A flow chart of said method is illustrated in FIG. 3. Said method comprises the step of:
S1: Attaching a closure 1; 1′ according to the invention to the container 100; 100′; 100″, hereby providing a bioreactor 200; 200′; 200″. As described above, the container 100; 100′; 100″ can be an empty container, such as an ordinary bottle which is not specifically adapted for being a bioreactor.

The method may further comprise the step of:
S2: Adjusting a length of the impeller shaft 13 according to the size of the container 100; 100′; 100″

The bioreactor 200; 200′; 200″ can then be used for culturing cells according to well known processes. The bioreactor 200; 200′; 200″ can be connected to a control system for control of a process for culturing cells, such as control of gas supply (for example air, O₂, N₂ or CO₂), addition of other ingredients (for example nutrients, pH regulating agents or new cell culture media), sampling, etc., as described above, for example in dependence on sensor output. Such a control system is well known in the art and will not be further described here. Hereby for example pH and DO in the cell culture can be controlled.

What is claimed is:

1-14. (canceled)

15. A closure for a bioreactor configured for being connectable to a container such that the closure and the container together constitutes the bioreactor, wherein the closure comprises an inside surface which will face an inside of the bioreactor when the closure is connected to the container and an outside surface which will form a part of an outer surface of the bioreactor when the closure is connected to the container and wherein the closure comprises:
an impeller device comprising an impeller shaft and impeller blades mounted to the impeller shaft, wherein said impeller device is mounted to the inside surface of the closure such that the impeller shaft and the impeller blades protrude into the container when the closure is connected to the container;
a gas delivery device comprising a gas inlet, a gas outlet and a gas delivery channel reaching between said gas inlet and said gas outlet, wherein said gas inlet is provided at the outside surface of the closure and said gas delivery channel is arranged in the closure such that it passes from the gas inlet at the outside surface of the closure to the inside surface of the closure and protrudes from the inside surface such that the gas outlet of the gas delivery channel is provided inside the container when the closure is connected to the container; and
a sensor arrangement comprising one or more sensors located within at least one sensor accessing channel, which sensor arrangement is arranged in the closure such that the one or more sensors are provided inside the container when the closure is connected to the container, the at least one sensor accessing channel is connected to the impeller shaft.

16. A closure according to claim 15, wherein the impeller shaft is telescopic whereby a length of the impeller shaft can be adjusted.

17. A closure according to claim 16, wherein the impeller shaft comprises an hollow outer shaft which is connected to the inside surface of the closure and an inner shaft which is telescopically arranged inside the hollow outer shaft such that it can protrude from the hollow outer shaft to different lengths, wherein said inner shaft is connected to the impeller blades and wherein said inner shaft is mounted to the hollow outer shaft such that it can rotate around a longitudinal central axis, A, of the impeller shaft.

18. A closure according to claim 15, wherein the gas delivery channel of the gas delivery device is provided inside the impeller device.

19. A closure according to claim 18, wherein said sensor accessing channel is arranged in the closure such that it passes from the outside surface of the closure to the inside surface of the closure and protrudes from the inside surface of the closure and wherein the one or more sensors are arranged in the sensor accessing channel such that the sensors can be used for measuring features of a content provided in the bioreactor when the closure is connected to the container and such that the one or more sensors are accessible from the outside surface of the closure via the sensor accessing channel.

20. A closure according to claim 18, wherein the one or more sensors comprise a pH sensor and/or a DO sensor and wherein the sensors are optical sensors and/or chemo electrical sensors.

21. A closure according to claim 15, wherein the gas inlet is configured for connection to an external gas source.

22. A closure according to claim 15, wherein the impeller device comprises a magnetic part for allowing magnetic drive for rotation of the impeller device from outside the container when the closure is connected to the container.

23. A closure according to claim 15, wherein the closure further comprises a securing mechanism for securing the closure to the container.

24. A closure according to claim 15, wherein the closure is configured for being connected to a container which is a bottle.

25. A closure according to claim 15, wherein the closure is configured for being connected to a container having an inner volume of between 50 mL-2 L.

26. A bioreactor comprising a closure according to claim 15 and a container connected to each other.

27. A method for transforming a container into a bioreactor, said method comprising the step of:
attaching a closure according to claim 15 to the container thereby providing a bioreactor.

28. A method according to claim 27, further comprising the step of:
adjusting a length of the impeller shaft according to the size of the container.
